(19) United States
(12) Patent Application Publication
(10) Pub. No.: US 2025/0281543 A1
(43) Pub. Date: Sep. 11, 2025

(54) PREVENTION OF AUTOIMMUNE DIABETES

(71) Applicant: CREATIVE MEDICAL TECHNOLOGIES, INC., Phoenix, AZ (US)

(72) Inventors: Courtney Bartlett, Niceville, FL (US); Timothy Warbington, Phoenix, AZ (US); Amit Patel, Salt Lake City, UT (US)

(73) Assignee: CREATIVE MEDICAL TECHNOLOGIES, INC., Phoenix, AZ (US)

(21) Appl. No.: 19/045,363

(22) Filed: Feb. 4, 2025

Related U.S. Application Data
(60) Provisional application No. 63/561,672, filed on Mar. 5, 2024.

(51) Int. Cl. A61K 35/28; A61K 9/00; A61K 35/50; A61K 35/51; A61K 38/19; A61K 45/06; A61P 3/10

(52) U.S. Cl. CPC A61K 35/28; A61K 9/0019; A61K 35/50; A61K 35/51; A61K 38/19; A61K 45/06; A61P 3/10

(57) ABSTRACT

Methods of improving muscle receptor sensitivity to insulin muscle receptors, reducing death, destruction or rejection of beta islets in patients having insulin dependent diabetes, wherein said method comprising the steps of: a) obtaining a patient in need of immune cell therapy; b) administering to said patient immunomodulatory cells; c) assessing the prevention and transition to diabetes requiring insulin.

### CROSS-REFERENCE TO RELATED APPLICATIONS

The present application claims benefit of U.S. Provisional Patent Application Ser. No. 63/561,672, filed on Mar. 5, 2024 entitled PREVENTION OF AUTOIMMUNE DIABETES, the contents of which are incorporated herein by reference in its entirety.

### FIELD OF THE INVENTION

The invention pertains to the field of autoimmune type 2 diabetes. whereby preventing of diabetes through the administration of immunomodulatory cells from allogeneic, autologous, or xenogeneic sources. The invention pertains to the field of improving muscle receptor sensitivity to insulin along with increasing beta islet cell survival and reduction/prevention of immunological destruction of said cell and associated cells types. The invention relates to the field of regenerative medicine, more specifically, the invention pertains to the field of inducing regenerative processes to intervertebral disc that are undergoing loss of function, more specifically, the invention relates to treatment of disc degenerative disease using B regulatory cells

### BACKGROUND OF THE INVENTION

Autoimmune type 2 diabetes, also known as type 1.5 diabetes or latent autoimmune diabetes in adults (LADA), is a distinct form of diabetes that shares characteristics of both type 1 and type 2 diabetes. Unlike classic type 2 diabetes, which is primarily characterized by insulin resistance and relative insulin deficiency due to impaired pancreatic function, autoimmune type 2 diabetes involves an autoimmune attack on pancreatic beta cells, leading to their destruction and subsequent insulin deficiency. This autoimmune process is similar to that seen in type 1 diabetes, although it tends to occur more gradually and often in adulthood, hence the term “latent” autoimmune diabetes.

One of the hallmarks of autoimmune type 2 diabetes is the presence of autoantibodies targeting various proteins found in the pancreatic islet cells, including Islet Cell Antibodies (ICA), Insulin Autoantibodies (IAA), glutamic acid decarboxylase (GAD), insulinoma-associated antigen-2 (IA-2), and zinc transporter 8 (ZnT8). These autoantibodies are indicative of an immune-mediated attack on the pancreatic beta cells, leading to their destruction and eventual loss of insulin production. The presence of these autoantibodies distinguishes autoimmune type 2 diabetes from classic type 2 diabetes, where autoimmunity is not typically involved.

In addition to autoantibodies, there is growing evidence suggesting that dysfunction of muscle receptors may play a role in the pathogenesis of autoimmune type 2 diabetes. Muscle receptors, including insulin receptors and glucose transporters such as GLUT4, play a crucial role in regulating glucose uptake and utilization in muscle tissue. In autoimmune type 2 diabetes, impaired signaling through these receptors may contribute to insulin resistance and impaired glucose uptake by muscle cells, leading to hyperglycemia and worsening of diabetes symptoms.

The exact mechanisms underlying muscle receptor dysfunction in autoimmune type 2 diabetes are not fully understood, but it is believed to involve a combination of genetic, environmental, and immunological factors. Genetic predisposition may influence the expression and function of insulin receptors and glucose transporters, rendering individuals more susceptible to insulin resistance and glucose intolerance. Environmental factors such as obesity, physical inactivity, and dietary factors may further exacerbate muscle receptor dysfunction by promoting insulin resistance and impairing glucose metabolism.

Immunological factors related to autoimmunity may also contribute to muscle receptor dysfunction in autoimmune type 2 diabetes. Chronic inflammation and immune dysregulation associated with autoimmune processes may disrupt insulin signaling pathways and interfere with glucose uptake by muscle cells, contributing to insulin resistance and hyperglycemia. Additionally, cross-reactivity between autoantibodies targeting pancreatic islet cells and muscle receptors has been proposed as a potential mechanism linking autoimmunity to muscle dysfunction in autoimmune type 2 diabetes.

Management of autoimmune type 2 diabetes involves a multifaceted approach aimed at addressing both autoimmune-mediated pancreatic beta cell destruction and muscle receptor dysfunction. Treatment may include immunomodulatory therapies to suppress autoimmune activity, insulin therapy to replace deficient insulin production, and lifestyle interventions such as diet and exercise to improve insulin sensitivity and glucose control.

Cell therapy has been utilized to treat diabetes after it progressed but has not been used to prevent disease.

### SUMMARY OF THE INVENTION

Preferred embodiments are drawn to methods of improving muscle receptor sensitivity to insulin muscle receptors, reducing death, destruction or rejection of beta islets in patients having insulin dependent diabetes, wherein said method comprising the steps of: a) obtaining a patient in need of immune cell therapy; b) administering to said patient immunomodulatory cells; c) assessing the prevention and transition to diabetes requiring insulin.

A summary of the invention is provided below with respect to the following numbered aspects.

1. A method for preventing type 2 autoimmune diabetes in patients with one or more autoantibodies targeting islet cell antibodies (ICA), comprising administering perinatal cell therapy.

2. The method of aspect 1, wherein the perinatal cell therapy is administered to patients with autoantibodies including Insulin Autoantibodies (IAA).

3. The method of aspect 1, wherein the perinatal cell therapy is administered to patients with autoantibodies including glutamic acid decarboxylase (GAD).

4. The method of aspect 1, wherein the perinatal cell therapy is administered to patients with autoantibodies including insulinoma-associated antigen-2 (IA-2).

5. The method of aspect 1, wherein the perinatal cell therapy is administered to patients with autoantibodies including zinc transporter 8 (ZnT8).

6. The method of aspect 1, wherein the perinatal cell therapy comprises mesenchymal stem cells derived from perinatal tissue.

7. The method of aspect 1, wherein the perinatal cell therapy comprises hematopoietic stem cells derived from perinatal tissue.

8. The method of aspect 1, wherein the perinatal cell therapy comprises amniotic fluid-derived stem cells.

9. The method of aspect 1, wherein the perinatal cell therapy comprises umbilical cord blood/tissue-derived stem cells.

10. The method of aspect 1, wherein the perinatal cell therapy comprises placental-derived stem cells.

11. The method of aspect 1, wherein the perinatal cell therapy comprises perinatal tissue-derived cells selected from the group consisting of Wharton's jelly cells, amniotic epithelial cells, umbilical cord tissue and chorionic plate-derived cells.

12. The method of aspect 1, wherein the perinatal cell therapy is administered via intravenous injection.

13. The method of aspect 1, wherein the perinatal cell therapy is administered via intramuscular injection.

14. The method of aspect 1, wherein the perinatal cell therapy is administered via subcutaneous injection.

15. The method of aspect 1, wherein the perinatal cell therapy is administered via intra-pancreatic injection.

16. The method of aspect 1, wherein the perinatal cell therapy is administered via intra-arterial injection.

17. The method of aspect 1, wherein the perinatal cell therapy is administered via intrathecal injection.

18. The method of aspect 1, wherein the perinatal cell therapy is administered via direct implantation into pancreatic tissue.

19. The method of aspect 1, further comprising administering a carrier solution with the perinatal cell therapy.

20. The method of aspect 19, wherein the carrier solution comprises a saline solution.

21. The method of aspect 19, wherein the carrier solution comprises a biocompatible hydrogel.

22. The method of aspect 1, further comprising administering immunosuppressive agents before the perinatal cell therapy such as teplizumab or rhGAD65.

23. The method of aspect 1, further comprising administering anti-inflammatory agents along with the perinatal cell therapy.

24. The method of aspect 1, further comprising administering growth factors along or before the perinatal cell therapy such as GABA.

25. The method of aspect 1, further comprising administering cytokines along with the perinatal cell therapy.

26. The method of aspect 1, wherein the efficacy of the perinatal cell therapy is assessed by monitoring levels of HbA1c.

27. The method of aspect 1, wherein the efficacy of the perinatal cell therapy is assessed by monitoring levels of autoantibodies.

28. The method of aspect 1, wherein the efficacy of the perinatal cell therapy is assessed by monitoring levels of c-peptide.

29. The method of aspect 1, wherein the efficacy of the perinatal cell therapy is assessed by conducting glucose tolerance tests.

30. The method of aspect 1, wherein the efficacy of the perinatal cell therapy is assessed by monitoring weight changes.

31. The method of aspect 1, wherein the efficacy of the perinatal cell therapy is assessed by monitoring renal function.

32. The method of aspect 1, wherein the efficacy of the perinatal cell therapy is assessed by monitoring cardiac function.

33. The method of aspect 1, wherein the efficacy of the perinatal cell therapy is assessed by monitoring retinopathy.

34. The method of aspect 1, wherein the efficacy of the perinatal cell therapy is assessed by evaluating the Clark score.

35. The method of aspect 1, wherein the efficacy of the perinatal cell therapy is assessed by monitoring sarcopenia.

36. The method of aspect 1, wherein the efficacy of the perinatal cell therapy is assessed by evaluating frailty.

37 A pharmaceutical composition for preventing type 2 autoimmune diabetes, comprising perinatal cells and a carrier solution.

38. The pharmaceutical composition of aspect 37, wherein the perinatal cells are selected from the group consisting of mesenchymal stem cells, hematopoietic stem cells, amniotic fluid-derived stem cells, umbilical cord blood-derived stem cells, and placental-derived stem cells.

39. The pharmaceutical composition of aspect 37, wherein the carrier solution is selected from the group consisting of saline solution and biocompatible hydrogel.

40. A kit for preventing type 2 autoimmune diabetes, comprising a container housing perinatal cells, a carrier solution, and instructions for administration.

41. The kit of aspect 40, wherein the perinatal cells are selected from the group consisting of mesenchymal stem cells, hematopoietic stem cells, amniotic fluid-derived stem cells, umbilical cord blood-derived stem cells, and placental-derived stem cells.

42. The kit of aspect 40, wherein the carrier solution is selected from the group consisting of saline solution and biocompatible hydrogel.

43. The kit of aspect 40, further comprising immunosuppressive agents.

44. The kit of aspect 40, further comprising anti-inflammatory agents.

45. The kit of aspect 40, further comprising growth factors.

46. The kit of aspect 40, further comprising cytokines.

47. A method of assessing the efficacy of perinatal cell therapy in preventing type 2 autoimmune diabetes, comprising monitoring levels of HbA1c.

48. The method of aspect 47, further comprising monitoring levels of autoantibodies.

49. The method of aspect 47, further comprising monitoring levels of c-peptide.

50. The method of aspect 47, further comprising conducting glucose tolerance tests.

51. The method of aspect 47, further comprising monitoring weight changes.

52. The method of aspect 47, further comprising monitoring renal function.

53. The method of aspect 47, further comprising monitoring cardiac function.

54. The method of aspect 47, further comprising monitoring retinopathy.

55. The method of aspect 47, further comprising evaluating the Clark score.

56. The method of aspect 47, further comprising monitoring sarcopenia.

57. The method of aspect 47, further comprising evaluating frailty.

58. A method of preventing type 2 autoimmune diabetes in a patient at risk, comprising administering perinatal cell therapy and assessing the efficacy of the therapy through one or more independent means selected from the group consisting of monitoring levels of HbA1c, autoantibodies, c-peptide, conducting glucose tolerance tests, monitoring weight changes, renal function, cardiac function, retinopathy, Clark score, sarcopenia, and frailty.

59. The method of aspect 58, wherein the perinatal cell therapy comprises mesenchymal stem cells derived from perinatal tissue.

60. The method of aspect 58, wherein the perinatal cell therapy comprises hematopoietic stem cells derived from perinatal tissue.

61. The method of aspect 58, wherein the perinatal cell therapy comprises amniotic fluid-derived stem cells.

62. The method of aspect 58, wherein the perinatal cell therapy comprises umbilical cord blood-derived stem cells.

63. The method of aspect 58, wherein the perinatal cell therapy comprises placental-derived stem cells.

64. The method of aspect 58, wherein the perinatal cell therapy comprises perinatal tissue-derived cells selected from the group consisting of Wharton's jelly cells, amniotic epithelial cells, and chorionic plate-derived cells.

65. The method of aspect 58, wherein the perinatal cell therapy is administered via intravenous injection.

66. The method of aspect 58, wherein the perinatal cell therapy is administered via intramuscular injection.

67. The method of aspect 58, wherein the perinatal cell therapy is administered via subcutaneous injection.

68. The method of aspect 58, wherein the perinatal cell therapy is administered via intra-pancreatic injection.

69. The method of aspect 58, wherein the perinatal cell therapy is administered via intra-peritoneal injection.

70. The method of aspect 58, wherein the perinatal cell therapy is administered via intrathecal injection.

71. The method of aspect 58, wherein the perinatal cell therapy is administered via direct implantation into pancreatic tissue.

72. The method of aspect 58, further comprising administering a carrier solution with the perinatal cell therapy.

73. The method of aspect 72, wherein the carrier solution comprises a saline solution.

74. The method of aspect 72, wherein the carrier solution comprises a biocompatible hydrogel.

75. The method of aspect 58, further comprising administering immunosuppressive agents along with the perinatal cell therapy.

76. The method of aspect 58, further comprising administering anti-inflammatory agents along with the perinatal cell therapy.

77. The method of aspect 58, further comprising administering growth factors along with the perinatal cell therapy.

78. The method of aspect 58, further comprising administering cytokines along with the perinatal cell therapy.

79. The method of aspect 58, wherein the perinatal cell therapy is administered in conjunction with insulin therapy.

80. The method of aspect 58, wherein the perinatal cell therapy is administered in conjunction with diet and exercise modifications.

81. The method of aspect 58, wherein the perinatal cell therapy is administered in conjunction with other diabetes preventive measures.

82. The method of aspect 58, wherein the perinatal cell therapy is administered in conjunction with genetic screening.

83. The method of aspect 58, wherein the perinatal cell therapy is administered in conjunction with lifestyle counseling.

84. The method of aspect 58, wherein the perinatal cell therapy is administered in conjunction with regular monitoring and follow-up.

85. The method of aspect 58, wherein the perinatal cell therapy is administered in conjunction with personalized treatment plans.

86. The method of aspect 58, wherein the perinatal cell therapy is administered in a hospital or clinical setting.

87. The method of aspect 58, wherein the perinatal cell therapy is administered in an outpatient setting.

88. The method of aspect 58, wherein the perinatal cell therapy is administered under the supervision of a healthcare professional.

89. The method of aspect 58, wherein the perinatal cell therapy is administered according to a predetermined dosage regimen.

90. The method of aspect 58, wherein the perinatal cell therapy is administered as a single dose.

91. The method of aspect 58, wherein the perinatal cell therapy is administered as multiple doses.

92. The method of aspect 58, wherein the perinatal cell therapy is administered at predetermined intervals.

93. The method of aspect 58, wherein the perinatal cell therapy is administered based on patient-specific factors.

94. The method of aspect 58, wherein the perinatal cell therapy is administered in combination with other therapeutic interventions.

95. The method of aspect 58, wherein the perinatal cell therapy is administered sequentially with other treatments.

96. The method of aspect 58, wherein the perinatal cell therapy is administered concurrently with other treatments.

97. The method of aspect 58, wherein the perinatal cell therapy is administered as a standalone treatment.

98. The method of aspect 58, further comprising monitoring the patient for adverse effects following perinatal cell therapy administration.

99. The method of aspect 98, wherein adverse effects are managed accordingly.

100. The method of aspect 58, wherein the patient's response to perinatal cell therapy is monitored over time.

101. The method of aspect 58, further comprising adjusting the treatment plan based on the patient's response.

102. The method of aspect 58, further comprising titrating the dosage of perinatal cell therapy based on efficacy and safety considerations.

103. The method of aspect 58, wherein the patient's medical history is considered in determining the suitability of perinatal cell therapy.

104. The method of aspect 58, further comprising obtaining informed consent from the patient prior to perinatal cell therapy administration.

105. The method of aspect 58, wherein the patient is informed about the potential benefits and risks of perinatal cell therapy.

106. The method of aspect 58, further comprising educating the patient about the importance of adherence to the treatment plan.

107. The method of aspect 58, wherein the patient is provided with resources for support and guidance during the treatment process.

108. The method of aspect 58, further comprising documenting the patient's response to perinatal cell therapy for future reference.

109. The method of aspect 58, wherein the patient's progress is regularly reviewed and documented by healthcare professionals.

110. The method of aspect 58, wherein the efficacy of perinatal cell therapy in preventing type 2 autoimmune diabetes is confirmed through clinical trials and long-term follow-up studies.

111. A method for preventing type 2 autoimmune diabetes in a patient at risk thereof, comprising administering perinatal cell therapy in combination with pancreatic islet transplantation to restore insulin production and glucose regulation.

112. The method of aspect 111, wherein the pancreatic islet transplantation is performed using allogeneic or xenogeneic islets.

113. A method for preventing type 2 autoimmune diabetes in a patient at risk thereof, comprising administering perinatal cell therapy in combination with gene therapy to modulate immune responses and promote tolerance to pancreatic antigens.

114. The method of aspect 113, wherein the gene therapy involves the delivery of genes encoding immunomodulatory proteins such as IL-10 or TGF-beta.

115. A method for preventing type 2 autoimmune diabetes in a patient at risk thereof, comprising administering perinatal cell therapy in combination with regenerative medicine approaches to repair damaged pancreatic tissue and promote beta-cell regeneration.

116. The method of aspect 115, wherein the regenerative medicine approaches involve the use of growth factors, stem cells, or tissue engineering scaffolds.

117. A method for preventing type 2 autoimmune diabetes in a patient at risk thereof, comprising administering perinatal cell therapy in combination with vaccination strategies to induce immune tolerance and prevent autoimmune destruction of pancreatic islet cells.

118. The method of aspect 117, wherein the vaccination strategies involve the administration of peptides derived from pancreatic antigens or immunomodulatory adjuvants.

119. A method for preventing type 2 autoimmune diabetes in a patient at risk thereof, comprising administering perinatal cell therapy in combination with anti-inflammatory agents to reduce systemic inflammation and autoimmune responses.

120. The method of aspect 119, wherein the anti-inflammatory agents are selected from the group consisting of NSAIDs, corticosteroids, and biologic agents targeting pro-inflammatory cytokines.

121. A method for preventing type 2 autoimmune diabetes in a patient at risk thereof, comprising administering perinatal cell therapy in combination with dietary supplements or nutraceuticals to promote pancreatic health and insulin sensitivity.

122. The method of aspect 121, wherein the dietary supplements or nutraceuticals include omega-3 fatty acids, vitamin D, and antioxidant compounds.

123. A method for preventing type 2 autoimmune diabetes in a patient at risk thereof, comprising administering perinatal cell therapy in combination with exercise regimens to improve glucose metabolism and insulin sensitivity.

124. The method of aspect 123, wherein the exercise regimens include aerobic exercise, resistance training, and flexibility exercises tailored to the individual patient's needs and abilities.

125. A method for preventing type 2 autoimmune diabetes in a patient at risk thereof, comprising administering perinatal cell therapy in combination with stress reduction techniques to mitigate the effects of chronic stress on glucose regulation and immune function.

126. The method of aspect 125, wherein the stress reduction techniques include mindfulness meditation, yoga, and cognitive-behavioral therapy.

127. A method for preventing type 2 autoimmune diabetes in a patient at risk thereof, comprising administering perinatal cell therapy in combination with sleep hygiene practices to optimize sleep quality and duration, which are known to affect glucose metabolism and immune function.

128. The method of aspect 127, wherein the sleep hygiene practices include maintaining a regular sleep schedule, creating a comfortable sleep environment, and avoiding stimulants such as caffeine before bedtime.

129. A method for preventing type 2 autoimmune diabetes in a patient at risk thereof, comprising administering perinatal cell therapy in combination with smoking cessation programs to reduce the risk of vascular complications and systemic inflammation associated with tobacco use.

130. The method of aspect 127, wherein the smoking cessation programs include behavioral counseling, nicotine replacement therapy, and prescription medications such as varenicline or bupropion.

131. A method for preventing type 2 autoimmune diabetes in a patient at risk thereof, comprising administering perinatal cell therapy in combination with lipid-lowering agents to control dyslipidemia and reduce the risk of cardiovascular disease, which is commonly associated with diabetes.

132. The method of aspect 131, wherein the lipid-lowering agents include statins, fibrates, and PCSK9 inhibitors.

133. A method for preventing type 2 autoimmune diabetes in a patient at risk thereof, comprising administering perinatal cell therapy in combination with blood pressure management strategies to prevent hypertension and reduce the risk of cardiovascular complications.

134. The method of aspect 133, wherein the blood pressure management strategies include lifestyle modifications such as salt restriction, weight loss, and regular exercise, as well as pharmacological interventions such as ACE inhibitors, ARBs, and diuretics.

135. A method for preventing type 2 autoimmune diabetes in a patient at risk thereof, comprising administering perinatal cell therapy in combination with kidney protective agents to preserve renal function and reduce the risk of diabetic nephropathy.

136. The method of aspect 135, wherein the kidney protective agents include angiotensin-converting enzyme (ACE) inhibitors, angiotensin II receptor blockers (ARBs), and sodium-glucose cotransporter-2 (SGLT2) inhibitors.

137. A method for preventing type 2 autoimmune diabetes in a patient at risk thereof, comprising administering perinatal cell therapy in combination with foot care education and preventive measures to reduce the risk of diabetic foot ulcers and lower extremity amputations.

138. The method of aspect 137, wherein the foot care education includes proper foot hygiene, regular inspection of the feet for signs of injury or infection, and the use of appropriate footwear to minimize pressure and friction on vulnerable areas of the feet.

139. A method for preventing type 2 autoimmune diabetes in a patient at risk thereof, comprising administering perinatal cell therapy in combination with eye care interventions to prevent diabetic retinopathy and other ocular complications associated with diabetes.

140. The method of aspect 139, wherein the eye care interventions include regular eye examinations by an ophthalmologist or optometrist, blood sugar control, and treatment of retinopathy with laser therapy or intravitreal injections of anti-VEGF agents.

141. A method for preventing type 2 autoimmune diabetes in a patient at risk thereof, comprising administering perinatal cell therapy in combination with mental health support services to address the psychological impact of living with diabetes and reduce the risk of depression and anxiety.

142. The method of aspect 141, wherein the mental health support services include counseling, psychotherapy, and support groups for individuals with diabetes and their families.

143. A method for preventing type 2 autoimmune diabetes in a patient at risk thereof, comprising administering perinatal cell therapy in combination with reproductive health counseling and family planning services to address the unique needs and concerns of individuals with diabetes of childbearing age.

144. The method of aspect 143, wherein the reproductive health counseling includes guidance on achieving glycemic control before and during pregnancy, the management of diabetes-related complications during pregnancy, and the use of contraception to prevent unplanned pregnancies in individuals with diabetes.

145. A method for preventing type 2 autoimmune diabetes in a patient at risk thereof, comprising administering perinatal cell therapy in combination with education and support services to promote adherence to medication regimens, self-monitoring of blood sugar levels, and other aspects of diabetes self-management.

146. The method of aspect 145, wherein the education and support services include individualized counseling sessions with a diabetes educator, group classes on diabetes management, and access to educational materials such as brochures, videos, and online resources.

147. A method for preventing type 2 autoimmune diabetes in a patient at risk thereof, comprising administering perinatal cell therapy in combination with telemedicine technologies to facilitate remote monitoring of blood sugar levels, medication adherence, and other aspects of diabetes management.

148. The method of aspect 146, wherein the telemedicine technologies include smartphone apps, wearable devices for continuous glucose monitoring, and secure online portals for communicating with healthcare providers and accessing medical records.

### DETAILED DESCRIPTION OF THE INVENTION

The invention provides the use of B regulatory cells for treatment of disc degenerative disease. Broadly speaking, the invention describes the administration of B regulatory cells as a means of suppressing localized inflammation in the intervertebral disc, as well as in the perispinal space. By reducing inflammation, the B regulatory cells provide an environment conducive to regeneration. For example, the administration of B regulatory cells allows for activation and function of endogenous stem cells, as well as exogenously administered stem cells. The invention further provides for the administration of B regulatory cells as a stimulatory of angiogenesis. In one embodiment the invention provides the previously unknown ability of B regulatory cells to stimulate angiogenesis. Stimulation of angiogenesis in the perispinal area is important for the treatment of conditions such as lumbar ischemia, which is believed to be one of the causes of disc degeneration and/or disc pain.

D Type 2 diabetes mellitus (T2DM) poses a significant global health burden, characterized by insulin resistance, impaired insulin secretion, and chronic hyperglycemia. Conventional therapies often focus on managing symptoms rather than addressing underlying mechanisms. However, emerging research suggests that perinatal cell therapy holds promise for improving glucose management and mitigating complications of T2DM, particularly in patients with autoantibodies. The goal is identify and treat patients before their autoantibodies destroy their beta islet cells and insulin muscle receptors by utilizing perinatal cell therapy.

Perinatal Cell Therapy in T2DM Management: Perinatal cells, derived from tissues such as the umbilical cord, placenta, and amniotic fluid, possess unique regenerative and immunomodulatory properties. These cells exhibit multilineage differentiation potential, making them ideal candidates for cell-based therapies. In the context of T2DM, perinatal cell therapy offers a novel approach by targeting both insulin resistance and beta cell dysfunction, key pathophysiological features of the disease.

Glucose Management Enhancement: One of the primary mechanisms through which perinatal cell therapy improves glucose management in T2DM is by enhancing GLUT-4 sensitivity. GLUT-4 is a glucose transporter predominantly expressed in insulin-sensitive tissues such as skeletal muscle and adipose tissue. Insulin resistance leads to impaired translocation of GLUT-4 to the cell membrane, resulting in reduced glucose uptake. Studies have shown that perinatal cells can modulate insulin signaling pathways, promoting GLUT-4 translocation and enhancing glucose uptake by target tissues. This effect contributes to improved glycemic control and insulin sensitivity in T2DM patients receiving perinatal cell therapy.

Beta Islet Survival: In addition to enhancing insulin sensitivity, perinatal cell therapy also exerts protective effects on pancreatic beta cells, the primary producers of insulin. Beta cell dysfunction and apoptosis contribute to progressive beta cell loss in T2DM, exacerbating hyperglycemia. Perinatal cells secrete factors such as growth factors, cytokines, and extracellular vesicles, which promote beta cell survival and proliferation. These paracrine effects create a favorable microenvironment within the pancreatic islets, preserving beta cell mass and function. Furthermore, perinatal cells possess immunomodulatory properties, suppressing autoimmune responses against beta cells in T2DM patients with autoantibodies. By preserving beta islet integrity, perinatal cell therapy offers a long-term solution for maintaining insulin secretion and glycemic control in T2DM.

Autoantibody-Mediated Mechanisms: Patients with T2DM often exhibit autoimmune features, including the presence of autoantibodies targeting pancreatic beta cells. These autoantibodies contribute to beta cell destruction and exacerbate disease progression. Perinatal cell therapy modulates the immune response through various mechanisms, including the induction of regulatory T cells (Tregs) and the suppression of pro-inflammatory cytokines. By promoting immune tolerance and dampening autoimmunity, perinatal cells mitigate the destructive effects of autoantibodies on beta cells, thereby preserving insulin secretion and preventing further deterioration of glycemic control.

Perinatal cell therapy represents a novel therapeutic approach for enhancing glucose management and mitigating complications of T2DM, especially in patients with autoantibodies. Through mechanisms such as GLUT-4 sensitivity enhancement and beta islet survival promotion, perinatal cells offer a multifaceted strategy for addressing the underlying pathophysiology of T2DM.

The invention, in some embodiments, teaches the application of immunomodulation and regeneration in patients with autoantibodies before they become insulin dependent diabetics. It is known that the immune system allows for recognition and elimination of pathological threats, while selectively ignoring antigens that belong to the body. Traditionally, autoimmune conditions or conditions associated with cytokine storm, or allograft rejection are treated with non-specific inhibitors of inflammation such as steroids, as well as immune suppressive agents such as cyclosporine, 5-azathrioprine, and methotrexate. These approaches globally suppress immune functions and have numerous undesirable side effects. Unfortunately, given the substantial decrease in quality of life observed in patients with autoimmunity, the potential of alleviation of autoimmune symptoms outweighs the side effects such as opportunistic infections and increased predisposition to neoplasia. However, in a patient who progress to diabetes, immunosuppressants lead to progression of disease and lead to end organ damage. Therefore, the goal is to treat the immune system before lead to long term damage, as in this case destruction of the functional capacity of the pancreas to produce insulin and muscle tissue to have functional insulin receptors (GLUT-4).

It should be noted that, as used in this specification and the appended claims, the singular forms “a,” and, “the” include plural referents unless the context clearly dictates otherwise. Thus, for example, reference to “a cell” includes one or more of such cells and reference to “the flask” includes reference to one or more of such flasks.

As used herein, the term “isolated cell” refers to a cell that has been isolated from a tissue, including from other cells of that tissue.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

As used herein, the definition of the term “stem cells or perinatal cells” relates to a cell capable of making copies of itself and having the capacity to differentiate into other types of cells. In some situations, “stem cells” is associated with research that may help patients suffering from previously incurable diseases. In other situations, “stem cells” represent “repair cells” of the body.

This same principle applies to ranges reciting only one numerical value as a minimum or a maximum. Furthermore, such an interpretation should apply regardless of the breadth of the range or the characteristics being described.

An initial overview of embodiments is provided below, and specific embodiments are then described in further detail. This initial summary is intended to aid readers in understanding the disclosure more quickly and is not intended to identify key or essential technological features, nor is it intended to limit the scope of the example subject matter.

Disclosed are perinatally tissue derived immunomodulatory cells and methods of extraction, expansion and potentiation therapeutic activity of said cells. In some cases, submembrane derived cells from perinatal tissue are isolated and expanded under physiological or disease-associated conditions ex vivo in order to retain or enhance therapeutic properties prior to administration for treatment of degenerative and/or autoimmune conditions.

To those of skill in the art, it has become known that there exist numerous types of stem cells, there are hematopoietic stem cells, which are responsible for production of blood. There are embryonic stem cells, which are capable of generating any tissue in the body, however, suffer from the predisposition to generate tumors termed “teratomas”. There are tissue-specific stem cells, such as brain stem cells in the dentate gyrus and subventricular zone, whose proliferation is associated with higher mental activity and resistance to depression. In one specific example, a stem/perinatal cell can be a mesenchymal stem cell (MSC).

While other reasoning is contemplated, MSCs can be useful due to a) MSCs are true “repair cells”. In many conditions, healing is associated with MSCs producing growth factors, which coordinate numerous cell types to initiate and maintain recovery of bodily function after injury; b) MSCs do not require matching between donor and recipient. This can be important because it allows for the use of the stem cell as a therapeutic, similar to an active agent or drug. A such, a large quantity of cells can be grown, standardized, characterized and subsequently used to treat a variety of different patients; c) MSCs act as “biological anti-inflammatoires”. Specifically, not only do MSC promote healing of injured tissues, but they also reduce inflammation. One very important aspect of MSC reducing inflammation is that they only reduce inflammation in the presence of tissue injury. This means that if MSCs are injected into a patient with no inflammation, then the MSCs do not produce anti-inflammatory products. In other cases, when a patient suffers from an inflammatory condition, the MSCs actually produce more anti-inflammatory agents in order to reduce the inflammation. Essentially, the MSC act as a “natural” anti-inflammatory cell, specifically “knowing” how many and what factors to producing; d) MSCs home to tissues of injury. It is known that most injury is associated with generation of blood clotting. This process results in lack of oxygen to the damaged tissue. When tissue lacks oxygen, cells of the tissue start producing “chemokines” which act as local beacons, calling in MSCs only to the area of tissue injury.

In addition to conceptual advantages of MSC, one useful aspect of this particular family of stem cells is that they have been used in numerous clinical trials with an excellent safety profile. While not all clinical trials have shown specific improvements from an efficacy perspective, adverse reactions to MSC administration, whether intravenous, intrathecal, intra-arterial, intramuscular, or the like, have largely not been observed.

Unfortunately, there appears to be room for improvement in the area of stem cells in general, and particularly in the area of mesenchymal stem cells. We believe that types of mesenchymal stem cells are needed because of: a) need for enhanced growth factor production by MSC; b) need for augmentation of homing by MSC to tissue of injury; and c) desire for MSC to be more economical. Here we describe the previous type of MSC from perinatal tissue, which have been modified to possess therapeutic activity that is superior to other stem cell types.

Those skilled in the art recognize numerous types of stem cells, including, for example, hematopoietic stem cells, which are responsible for the production of blood, tissue-specific stem cells, such as brain stem cells in the dentate gyrus and subventricular zone, whose proliferation is associated with higher mental activity and resistance to depression. Another example includes embryonic stem cells, which are capable of generating any tissue in the body; however, they suffer from the predisposition to generate tumors termed “teratomas.”

While numerous types of stem cells have been discovered, and most likely, many will be discovered, one type of useful class of stem cells are mesenchymal stem cells (MSCs). One reason for using MSCs as compared to other cell types for development of therapeutics is due to the fact that MSCs are true “repair cells.” In many conditions, healing is associated with MSCs producing growth factors, which coordinate numerous cell types to initiate and maintain recovery of bodily function after injury. Another reason is due to the fact that MSC's do not require matching between donor and recipient. This is important because it allows for the use of the stem cell as an “active agent,” such as a “drug” or other therapeutic agent. As such, large quantities MSCs can be grown, standardized, characterized, and subsequently used to treat a variety of different patients. In yet another reason, MSCs can act as “biological anti-inflammatories.” Specifically, not only do MSCs promote healing of injured tissues, but they also reduce inflammation. One important aspect of MSC reducing inflammation is that they only reduce inflammation in the presence of tissue injury. This means that if MSCs are injected into a patient with no inflammation, then the MSCs do not produce anti-inflammatory products. In other cases, when the patient suffers from an inflammatory condition, the MSCs actually produce more anti-inflammatory agents in order to reduce the inflammation. Essentially, the MSCs act as “natural” anti-inflammatory cells, specifically “knowing” how many and what factors to produce. In a further example, MSCs can home in on injured tissues. It is known that most injury is associated with generation of blood clotting. This process results in lack of oxygen to the damaged tissue. When tissue lacks oxygen, cells of the tissue start producing “chemokines” which act as local beacons, calling in MSC only to the area of tissue injury.

In addition to conceptual advantages of MSC, one very important aspect of this particular family of stem cells is that they have been used in numerous clinical trials with an excellent safety profile. While not all clinical trials have shown specific improvements from an efficacy perspective, adverse reactions to MSC administration, whether intravenous, intrathecal, intra-arterial, or intramuscular, have largely not been observed.

The present disclosure provides use of novel stem cell types which includes techniques for deriving stem cells possessing immunomodulatory, immunomodulatory, anti-inflammatory, and increasing muscle sensitivity from perinatal tissue. Perinatal tissue can include tissues such as such as the placenta, umbilical cord, cord blood, amniotic fluid, and the like. In some examples, the manipulation of stem cell “potency” can be achieved through hypoxic manipulation, growth under non-xenogeneic conditions, as well as addition of epigenetic modulators.

The presently disclosed perinatal cells can be cultured under hypoxia, in one example, in order to induce and/or augment expression of chemokine receptors. One specific example of such a receptor is CXCR-4. One example population of stem cells includes perinatal tissue mesenchymal cells. A variety of techniques can be utilized to extract the isolated stem cells, and any such technique that allows such extraction without significant damage to the stem cells is considered to be within the present scope. In one example, a method of culturing stem cells from the mammalian perinatal tissue can include the dissection of perinatal tissue. In one specific example, perinatal tissue can be collected and washed to remove blood, Wharton's Jelly, and any other material associated with the submembrane layer. In yet another non-limiting example the cord tissue can be washed multiple times in a solution of Phosphate-Buffered Saline (PBS) such as Dulbecco's Phosphate-Buffered Saline (DPBS). In some examples, the PBS can include a platelet lysate (i.e. 10% PRP lysate of platelet lysate-liquid or lyophilized). Any remaining undesired portion of the perinatal tissue can then be removed and discarded. The remaining perinatal tissue can then be placed interior side down on a substrate with the tissue of interest being in contact with the substrate. Any size of perinatal tissue section can be placed interior side down on a substrate. In one example, the perinatal tissue section can be the same or substantially the same size as the substrate. In another example, the dissected perinatal tissue can be cut into smaller sections (e.g. 1-3 mm) which can be placed directly onto the substrate.

A variety of substrates are contemplated upon which the submembrane layer (SL) can be placed. In one aspect, for example, the substrate can be a polymeric material, such as, for example, a solid polymeric material. One example of a solid polymeric material can include a cell culture dish. The cell culture dish can be made of a cell culture treated plastic as is known in the art. In one specific example, the SL can be placed upon the substrate of the cell culture dish without any additional pretreatment to the cell culture treated plastic. In another example, the substrate can be a semi-solid cell culture substrate. Such a substrate can include, for example, a semi-solid culture medium including an agar or other gelatinous base material.

Following placement of the submembrane layer on the substrate, the submembrane layer is cultured in a suitable medium. In some examples, the culture media can be free of animal and human components or contaminants. The culture can then be cultured under either normoxic or hypoxic culture conditions for a period of time sufficient to establish primary cell cultures. (e.g. 3-7 days in some cases). After primary cell cultures have been established, the submembrane layer tissue is removed and discarded. Cells or stem cells are further cultured and expanded in larger culture flasks in either a normoxic or hypoxic culture conditions. While a variety of suitable cell culture media are contemplated, in one non-limiting example the media can be Dulbecco's Modified Eagle Medium (DMEM) glucose (500-6000 mg/mL) without phenol red, 1X glutamine, 1X. NEAA, and 0.1-20% PRP lysate or platelet lysate. Another example of suitable media can include a base medium of DMEM low glucose without phenol red, 1X. glutamine, 1X. NEAA, with or without 1000 units of heparin and 20% PRP lysate liquid or lyophilized platelet lysate. In another example, cells can be cultured directly onto a semi-solid substrate of DMEM low glucose without phenol red, 1X glutamine, 1X. NEAA, and 20% PRP lysate liquid or lyophilized platelet lysate. In a further example, culture media can include a low glucose medium (500-1000 mg/mL) containing 1X. Glutamine, 1X. NEAA, 1000 units of heparin. In some aspects, the glucose can be 1000-4000 mg/mL, and in other aspects the glucose can be high glucose at 4000-6000 mg/mL. These media can also include 0.1%- 20% PRP lysate liquid or lyophilized platelet lysate. In yet a further example, the culture medium can be a semi-solid with the substitution of acid-citrate-dextrose ACD in place of heparin, and containing low glucose medium (500-1000 mg/ml), intermediate glucose medium (1000-4000 mg/mL) or high glucose medium (4000-6000 mg/mL), and further containing 1X. Glutamine, 1X. NEAA, and 0.1%- 20% PRP lysate liquid or lyophilized platelet lysate. In some aspects, the cells can be derived, subcultured, and/or passaged using TrypLE. In another aspect, the cells can be derived, subcultured, and/or passaged without the use of TrypLE or any other enzyme that is xeno-free.

In other embodiments of the invention, purified populations of immunomodulatory cells can be obtained from human perinatal tissue. As used herein, “purified” means that at least 90% (e.g., 91, 92, 93, 94, 95, 96, 97, 98, or 99%) of the cells within the population are immunomodulatory cells. As used herein, “immunomodulatory cells” refers to mammalian cell. Within the context of the current invention immunomodulatory cells can be isolated from perinatal tissues obtained with informed consent. Typically, after a perinatal tissue is obtained in a hospital or clinic, the tissye is placed in a hypothermic preservation solution, such as FRS solution from Biolife Solutions (catalog #HTS-FRS) and stored at 4° C. To begin isolating perinatal tissue derived cells, the hypothermic preservation solution can be removed by washing in a buffer, such as Hank's basic salt solution, that is free of Mg2+, Ca2+, and phenol free. The perinatal tissue can be cut into cross sections in the presence of a buffer, and then the cross-sections can be cut longitudinally into two pieces while avoiding any venous or arterial tissue. If any blood is released into the buffer while cutting the cord, the contaminated buffer is replaced with fresh buffer. The longitudinal pieces of cord can be dissected to remove venous and arterial tissue such that the resulting cord lining (i.e., the gelatinous cord material) is substantially free of venous and arterial tissue. As used herein “substantially free of venous and arterial tissue” indicates that as much visible venous and arterial tissue has been removed as possible with manual dissection. The immunomodulatory cells can be obtained from the dissected submembrane lining by culturing the longitudinal pieces of submembrane lining on a fibronectin coated solid substrate (e.g., a plastic culture device such as a chambered slide or culture flask). The gelatinous surface of the cord lining can be placed in contact with the fibronectin coated solid substrate while the upper surface (i.e., the surface not in contact with the fibronectin coated solid substrate) can be covered with a solid substrate such as a coverslip. Low glucose (i.e.ltoreq.1 g/L glucose) growth medium can be added and the culture device incubated for a time sufficient for cells to migrate from the cord lining to the fibronectin coated solid substrate (e.g., 7 to 10 days). Unless otherwise indicated, cells are cultured at 37° C. in a standard atmosphere that includes 5% CO2. Relative humidity is maintained at about 100%. After have adhered to the surface of the fibronectin coated solid substrate, the coverslip can be removed, and the adhered cells can be washed in a buffer such as phosphate-buffered saline (PBS). A growth medium that can be used for culturing Immunomodulatory cells is low glucose Dulbecco's Modified Essential Media (DMEM) containing vitamins (choline chloride, D-Calcium pantothenate, Folic Acid, Nicotinamide, Pyridoxal hydrochloride, Riboflavin, Thiamine hydrochloride, and i-Inositol), and non-essential amino acids (glycine, L-alanine, L-Asparagine, L-Aspartic acid, L-Glutamic Acid, L-Proline, and L-Serine). Low glucose DMEM can be supplemented with 10% to 20% serum (e.g., fetal bovine serum (FBS) or human serum), one or more antibiotics (e.g., gentamycin, penicillin, or streptomycin), and glutamine or a stabilized dipeptide of L-alanyl-L-glutamine (e.g., GlutaMax from Invitrogen). In one embodiment, a growth medium can include low glucose DMEM containing vitamins and non-essential amino acids, 15% FBS, 1 to 3% antibiotic (e.g., 2% or 2X. gentamycin), and 0.7 to 1.5% (e.g., 1%) of glutamine or a stabilized dipeptide of L-alanyl-L-glutamine. Such a growth medium can be further supplemented with 1 to 100 ng/ml of a growth factor (e.g., basic fibroblast growth factor (bFGF), leukemia inhibitory factor (LIF), or epidermal growth factor (EGF).

In some embodiments, a growth medium further includes insulin, transferrin, selenium, and sodium pyruvate. A particularly useful growth medium can include low glucose DMEM containing vitamins and non-essential amino acids, 15% serum, 1 to 3% antibiotic (e.g., 2% or 2X. gentamycin), 0.7 to 1.5% of glutamine or a stabilized dipeptide of L-alanyl-L-glutamine (e.g., 1% or 1X. GlutaMax), 1 to 100 ng/ml of a growth factor (e.g., 10 ng/ml bFGF and 10 ng/ml LIF), 0.1 mg/ml to 100 mg/ml of insulin (10 mg/mL), 0.1 mg/mL to 100 mg/mL of transferrin (e.g., 0.55 mg/ml transferring), 0.1 μg/mL to 100 μg/mL selenium (e.g., 0.5 μg/ml selenium), and 0.5 to 1.5% sodium pyruvate (e.g., 1% sodium pyruvate). In some embodiments, such a growth medium further includes 0.05 μg/mL to 100 μg/ml of putrescine (e.g., 10 μg/mL putrescine) and 10 ng/ml of EGF. For embodiments in which an animal free medium is desired, human serum (e.g., 15% human serum) can be used in place of fetal bovine serum or PRP lysate liquid or lyophilized platelet lysate.

In some embodiments of the invention, it is necessary to subculture immunomodulatory cells, TrypZean (Sigma Chemical Co.) can be used to release cells from the solid substrate. The resulting cell suspension can be pelleted and washed with PBS, then seeded into cell culture flasks at approximately 1000 cells/cm2 in a growth medium. Clonal lines of immunomodulatory cells can be established by plating the cells at a high dilution and using cloning rings (e.g., from Sigma) to isolate single colonies originating from a single cell. Cells are obtained from within the cloning ring using trypsin then re-plated in one well of a multi-well plate (e.g., a 6-well plate). After cells reach >60% confluency (e.g., >70% confluency), the cells can be transferred to a larger culture flask for further expansion. Immunomodulatory cells can be assessed for viability, proliferation potential, and longevity using techniques known in the art. For example, viability can be assessed using trypan blue exclusion assays, fluorescein diacetate uptake assays, or propidium iodide uptake assays. Proliferation can be assessed using thymidine uptake assays or MTT cell proliferation assays. Longevity can be assessed by determining the maximum number of population doublings of an extended culture.

Immunomodulatory cells can be immunophenotypically characterized using known techniques. For example, the cells can be fixed (e.g., in paraformaldehyde), permeabilized, and reactive sites blocked (e.g., with serum albumin), then incubated with an antibody having binding affinity for a cell surface antigen. The antibody can be detectably labeled (e.g., fluorescently or enzymatically) or can be detected using a secondary antibody that is detectably labeled. In some embodiments, the cell surface antigens on Immunomodulatory cells can be characterized using flow cytometry and fluorescently labeled antibodies.

Immunomodulatory cells also can be characterized based on the expression of one or more genes. Methods for detecting gene expression can include, for example, measuring levels of the mRNA or protein of interest (e.g., by Northern blotting, reverse-transcriptase (RT)-PCR, microarray analysis, Western blotting, ELISA, or immunohistochemical staining)

Immunomodulatory (perinatal MSC) cells can be cryopreserved by suspending the cells (e.g., up to 5×106 cells/mL) in a cryopreservative such as dimethylsulfoxide (DMSO, typically 10%). In some embodiments, a freezing medium such as CryoStor from Biolife solutions is used to cryopreserve the cells. After adding cryopreservative, the cells can be frozen (e.g., to −90° C.). In some embodiments, the cells are frozen at a controlled rate (e.g., controlled electronically or by suspending the cells in a bath of 70% ethanol and placed in the vapor phase of a liquid nitrogen storage tank. When the cells are chilled to ×90° C., they can be placed in the liquid phase of the liquid nitrogen storage tank for long term storage. Cryopreservation can allow for long-term storage of these cells for therapeutic use.

These cells isolated according to the above methodology may be enriched for CXCR-4, such as (or such as about) 50%, 55%, 60%, 65%, 70%, 75%, 80%, 85%, 90%, 95%, 96%, 97%, 98%, 99%, or more of the population expressing CXCR-4, CD31, CD34, or any combination thereof. In addition, or alternatively, <1%, <2%, <3%, <4%, <5%, <6%, <7%, <8%, <9%, or <10% of the population of cells may express CD14 and/or CD45. The umbilical cord cells of the invention may further possess markers selected from STRO-1, CD105, CD54, CD56, CD106, HLA-I markers, vimentin, ASMA, collagen-1, fibronectin, LFA-3, ICAM-1, PECAM-1, E-selectin, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1, and a combination thereof. In particular embodiments, cells of the invention lack expression of CD90, CD105 and CD34 but possess CD56, and/or CD73 expression.

The population of cells may be allogeneic, autologous, or xenogenic to an individual, including an individual being administered the population of cells. In some embodiments, the population of cells are matched by mixed lymphocyte reaction matching.

In some embodiments, the population of cells is derived from tissue selected from the placental body, placenta, umbilical cord tissue with interleukin-10, and/or interleukin 35 expression. In some embodiments, the population of cells, the population of perinatal tissue cells, or the population of endothelial cells express hTERT and Oct-4 but does not express a STRO-1 marker.

In some embodiments, the population of cells, the population of perinatal tissue cells has an ability to undergo cell division in less than 36 hours in a growth medium. In some embodiments, the population of cells, the population of perinatal tissue cells has an ability to proliferate at a rate of 0.9-1.2 doublings per 36 hours in growth media. In some embodiments, the population of cells, the population of perinatal tissue cells has an ability to proliferate at a rate of 0.9, 1.0, 1.1, or 1.2 doublings per 36 hours in growth media. The population of cells, population of perinatal tissue cells may produce exosomes capable of inducing more than 50% proliferation when the exosomes are cultured with human perinatal tissue endothelial cells. The induction of proliferation may occur when the exosomes are cultured with the human perinatal tissue endothelial cells at a concentration of 10, 20, 30, 40, 50, 60, 70, 80, 90, 100, 110, 120, 130, 140, 150, 160, 170, 180, 190, 200, or more exosomes per cell. Exosomes produced by cells described herein, in some embodiments express CD9, CD63 and/or CD81.

In some embodiments, a population of cells, including a population of perinatal tissue derived cells alone, are administered to an individual, including an individual having and acute or chronic pathology, wherein the population of cells may be administered via any suitable route, including as non-limiting examples, intraarterially, intramuscularly and/or intravenously.

The clinically course of patients who have Type 2 non-insulin dependent diabetes but with positive autoantibodies similar to type 1 diabetes patients is devastating. The usual treatments of weight and diet management along with oral pharmacological therapy and sometimes GLP-1 agents may temporalize progression to requiring insulin due to beta cell destruction and further impairment in GLUT-4 muscle receptor sensitivity. However, there patients progress to requiring insulin much quicker in the range of 1 to 3 years with positive autoantibodies versus 5-10 years with no antibodies. So, it is paramount to identifying patients who could benefit from immunomodulatory therapy in the earlier part of their disease one antibodies are identified and before progression to requiring insulin for optimal glucose management and end-organ disease and destruction. Also, for monitoring patients optimal use of continuous glucose monitors and digital application development have been required. The team has developed a digital glucose log that patient can utilize on an iOS or android compatible device along with a traditional web-based module. The application enables rapid determination of outliers of glucose which can immediately sent to the patients' healthcare team to provide much more rapid correction and intervention hopefully before diabetic ketoacidosis sets in or a trip to the hospital emergency department. Once this application was developed a clinical trial was designed and approved to intraarterially infuse perinatal cells directly to the pancreas which would secondarily pass systemic and impact GLUT-4 receptors based on skeletal muscles through the body to allow better sensitivity and utilization of insulin. All patients screened have autoantibodies after they have been diagnosed with Type 2 diabetes but have not deteriorated to requiring insulin for optimal glucose management and control.

### Experimental Procedure: Intraarterial Delivery of Perinatal Cells in Patients With Type 2 Diabetes Autoantibodies

Objective: The objective of this experimental procedure was to assess the safety and efficacy of delivering perinatal cells via intraarterial injection to patients with type 2 diabetes and autoimmune antibodies who do not require insulin, Assessments were based on the change in beta islet function as assessed by C-peptide levels, HgA1C, glucose tolerance test, along with autoantibodies.

### Materials Required

Perinatal cells (obtained from Human GMP Cell Bank)
Catheters for intra-arterial delivery
Heparin
Sterile saline solution
Intravenous catheters
Blood collection tubes
Glucose solution for oral glucose tolerance test (OGTT)
Laboratory equipment for C-peptide level analysis
Laboratory equipment for autoantibody analysis

### Experimental Procedure

### Patient Selection

Patients diagnosed with who have detectable autoantibodies against pancreatic islet cells who do not require insulin and are stable medical therapy for 30 days.

Ensured patients meet inclusion criteria such as age, health status, and willingness to participate in the study.

Obtained informed consent from each patient after explaining the nature and potential risks and benefits of the procedure.

### Preparation of Perinatal Cells

Perinatal cells from Human GMP Cell Bank used in prior studies with adequate safety history were utilized

Processed the perinatal cells according to established protocols to obtain a suspension suitable for injection.

### Intraarterial Injection

Prepared the patient for the procedure in a sterile operating room or interventional radiology suite.

Administered local anesthesia at the site of arterial access (common femoral artery or radial artery).

Patient given adequate heparin to obtain ACT>200.

Inserted an catheter with guidewire into the artery under fluoroscopic or ultrasound guidance.

Injected 100M perinatal cells suspended in sterile saline solution directly into the artery at 10M cells per minute.

Monitored the patient closely for any signs of adverse reactions during and after the injection-allergic reactions, hematoma, infection, injury to artery or pancreas, stroke, death,

### Post-Injection Monitoring

Monitored vital signs, including blood pressure, heart rate, and oxygen saturation, continuously during the procedure and post-injection period.

Performed regular blood draws to assess C-peptide levels and autoantibody titers before and after the injection at specified intervals.

### Assessment of Beta Islet Function

Performed OGTT before and after the injection to evaluate glucose tolerance and insulin response.

Measured C-peptide levels in the blood samples collected to assess beta islet function.

Compared pre- and post-injection C-peptide levels and OGTT results to determine improvement in beta islet function.

### Evaluation of Autoantibodies

Analyzed blood samples collected before and after the injection to quantify autoantibody levels.

Determined the percentage reduction in autoantibody titers following the intraarterial delivery of perinatal cells.

### Data Analysis

Compiled and analyzed the data obtained from C-peptide measurements, OGTT results, and autoantibody assays.

Calculated the percentage improvement in beta islet function and reduction in autoantibody titers compared to baseline levels.

Performed statistical analysis to determine the significance of the observed changes.

### Follow-Up

Scheduled follow-up appointments with patients to monitor their clinical status and any potential adverse effects.

Repeated assessments of beta islet function and autoantibody levels at regular intervals to evaluate the long-term effects of the treatment.

### Documentation

Maintained detailed records of each patient's medical history, procedure details, and post-treatment outcomes.

Documented any adverse events or complications encountered during the study.

### Conclusion

The experimental procedure outlined above aims to evaluate the efficacy of intraarterial delivery of perinatal cells in patients with type 2 diabetes but with antibodies. By assessing changes in beta islet function and other markers, the study seeks to provide valuable insights into the potential therapeutic benefits of this novel perinatal cell therapy approach.

### Summary of Clinical Data

There were 10 patients successfully consented, enrolled and treated. There were no severe adverse events over during the infusion and following up period. The only adverse event related to product or procedure during the infusion was a small bruise at the infusion catheter introduction site in two patients with no hematoma that resolved in 7 days. Results are shown in Table 1, below.

TABLE 1

| | | 1 | 3 | 6 | 12 |
|---|---|---|---|---|---|
| Parameter | Baseline | month | months | months | months |
| HgA1c | 7.7 ± | 7.6 ± | 6.5 ± | 6.6 ± | 6.3 ± |
| (mmol/mol) | 0.4 | 0.3 | 0.5 | 0.5 | 0.4 |
| Insulin(IU/kg/day) | 0 | 0 | 0 | 0 | 0 |
| Fasting C-peptide | 0.75 ± | 0.73 ± | 0.81 ± | 0.98 ± | 1.12 ± |
| (nmol/L) | 0.15 | 0.16 | 0.14 | 0.11 | 0.14 |

1. A method for preventing type 2 autoimmune diabetes in patients with one or more autoantibodies targeting islet cell antibodies (ICA), comprising administering perinatal cell therapy.

2. The method of claim 1, wherein the perinatal cell therapy comprises mesenchymal stem cells derived from perinatal tissue.

3. The method of claim 1, wherein the perinatal cell therapy comprises umbilical cord blood-derived stem cells.

4. The method of claim 1, wherein the perinatal cell therapy comprises placental-derived stem cells.

5. The method of claim 1, wherein the perinatal cell therapy is administered via intramuscular injection.

6. The method of claim 1, wherein the perinatal cell therapy is administered via intravascular injection.

7. The method of claim 1, wherein the perinatal cell therapy is administered via intra-pancreatic injection.

8. The method of claim 1, further comprising administering a carrier solution with the perinatal cell therapy.

9. The method of claim 8, wherein the carrier solution comprises a saline solution or a biocompatible hydrogel.

10. The method of claim 1, further comprising administering immunosuppressive agents and/or cytokines before the perinatal cell therapy.

11. The method of claim 1, further comprising administering anti-inflammatory agents along with the perinatal cell therapy.

12. The method of claim 1, further comprising administering growth factors before or along with the perinatal cell therapy.

13. The method of claim 1, wherein the efficacy of the perinatal cell therapy is assessed by monitoring levels of HbA1c, c-peptide, autoantibodies and/or Clark score.

14. The method of claim 1, wherein the efficacy of the perinatal cell therapy is assessed by monitoring frailty and sarcopenia.

15. A pharmaceutical composition for preventing type 2 autoimmune diabetes, comprising perinatal cells and a carrier solution.

16. The pharmaceutical composition of claim 15, wherein the perinatal cells are selected from mesenchymal stem cells, hematopoietic stem cells, amniotic fluid-derived stem cells, umbilical cord blood-derived stem cells, and placental-derived stem cells.

17. A kit for preventing type 2 autoimmune diabetes, comprising a container housing perinatal cells, a carrier solution, and instructions for administration.

18. A method for preventing type 2 autoimmune diabetes in a patient at risk thereof, comprising administering perinatal cell therapy in combination with pancreatic islet transplantation to restore insulin production and glucose regulation.

19. The method of claim 18, wherein the pancreatic islet transplantation is performed using allogeneic or xenogeneic islets.

20. A method for preventing type 2 autoimmune diabetes in a patient at risk thereof, comprising administering perinatal cell therapy in combination with gene therapy to modulate immune responses and promote tolerance to pancreatic antigens.
